(19) United States
(12) Patent Application Publication
(10) Pub. No.: US 2008/0090745 A1
(43) Pub. Date: Apr. 17, 2008

(54) Expression of Streptomyces subtilism inhibitor (SSI) proteins in Bacillus and Streptomyces sp.

(76) Inventors: Bryan P. Fox, Palo Alto, CA (US); Huaming Wang, Palo Alto, CA (US)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 11/580,948

(22) Filed: Oct. 13, 2006

(51) Int. Cl. C11D 3/386; C07H 21/04; C12P 21/06; C12N 9/56; C12N 1/21; C12N 15/74

(52) U.S. Cl. 510/320; 435/69.1; 435/222; 435/252.31; 435/471; 536/23.2; 435/252.35

(57) ABSTRACT

Streptomyces and Bacillus host cells comprising a recombinant nucleic acid encoding a fusion protein containing a signal sequence and a Streptomyces subtilisin inhibitor (SSI) protein are provided, as well as methods of producing SSI protein using those cells. In certain embodiments, the host cell is a Streptomyces host cell and the signal sequence is a celA signal sequence. In other embodiments, the host cell is a Bacillus host cell has a genome that contains inactivated protease genes.

### BACKGROUND

Proteases are found in many commercial products, with the largest application currently being their use in laundry detergents. However, many proteases are unstable and are known to undergo self-hydrolysis or autolysis. Such instability can limit the shelf-life of protease-containing products, particularly if the protease is active in the product.

A protease in a liquid laundry detergent may be stabilized in a number of different ways. For example, a protease in a liquid laundry detergent may be stabilized by small molecules such as a combination of 1,2-propanediol and boric acid. These molecules minimize autolytic protease activity in the undiluted laundry detergent and dissociate from the protease upon dilution of the laundry detergent use, allowing the protease to be active. In other cases, a protease in a liquid laundry detergent may be stabilized by a proteinaceous inhibitor of that protease. Proteinaceous protease inhibitors have advantages over small molecules protease inhibitors in that they can be co-produced with the protease being used, and further can be used at much lower concentrations than small molecules inhibitors.

This disclosure relates to the production of proteinaceous protease inhibitors known as Streptomyces subtilisin inhibitor protein (“SSI protein”). The SSI protein is an inhibitor of subtilisin, a protease that is commonly found in household detergent products.

### SUMMARY OF THE INVENTION

Streptomyces sp. and Bacillus sp. host cells comprising a recombinant nucleic acid encoding a fusion protein containing a signal sequence and a Streptomyces subtilisin inhibitor (SSI) protein are provided, as well as methods of producing SSI protein using those cells. In certain embodiments, the host cell is a Streptomyces host cell and recombinant nucleic acid includes a signal sequence that is a celA signal sequence. In other embodiments, the host cell is a Bacillus host cell having a genome that contains at least one inactivated protease gene.

In certain embodiments, a Streptomyces sp. host cell comprising: a recombinant nucleic acid encoding a fusion protein comprising: a) a celA signal sequence; and b) a Streptomyces Subtilisin Inhibitor (SSI) protein is provided. The cell may secrete the SSI protein from the cell into culture medium. In certain embodiments, the SSI protein may have increased stability in the presence of subtilisin and/or decreased affinity for subtilisin, as compared to a naturally-occurring SSI protein. In particular embodiments, the SSI protein may have an amino acid sequence that is at least 95% identical to SEQ ID NO:1, having a Lys at position 62, an Ile at position 63, a Pro at position 73, a Cys at position 83 and a Glu at position 98.

In certain embodiments, the celA signal sequence may be the signal sequence encoded by the celA gene of Streptomyces lividans. In particular embodiments, the celA signal sequence may comprise the amino acid sequence of SEQ ID NO:2.

The Streptomyces sp. host cell may be a S. lividans host cell which may, in certain cases, contain an inactivated ssi gene.

In certain embodiments, the recombinant nucleic acid may be operably linked to a promoter and terminator to form an expression cassette for the expression of the SSI protein. The recombinant nucleic acid may be codon optimized for expression of the SSI fusion protein in said Streptomyces sp. host cell. The recombinant nucleic acid may be present in the genome of the host cell or in vector that autonomously replicates in the cell.

A culture of cells comprising a plurality of the above-described Streptomyces sp. host cells and culture medium is also provided. The culture of cells in preferred embodiments comprises the SSI protein.

A method of producing an SSI protein, comprising culturing the above-described Streptomyces host cell to provide for secretion of the SSI protein in culture medium is provided. The method may further include recovering said SSI protein from said culture medium. The protein may be combined with a laundry detergent to produce a detergent that contains both a subtilisin protease and said SSI protein. The laundry detergent may be a borax-free laundry detergent.

In alternative embodiments, a Bacillus sp. host cell comprising: a recombinant nucleic acid encoding a fusion protein comprising: a) a signal sequence; and b) a Streptomyces Subtilisin Inhibitor (SSI) protein is provided. The host cell may secrete the SSI protein from the cell and the host cell may have inactivated aprE, nprE, epr, ispA, bpr, vpr, wprA, mpr-ybjF and/or nprB genes.

In certain embodiments, the SSI protein may have increased stability in the presence of subtilisin and/or decreased affinity for subtilisin, as compared to a naturally-occurring SSI protein. In particular embodiments, the protein may have an amino acid sequence that is at least 95% identical to SEQ ID NO:1, having a Lys at position 62, an Ile at position 63, a Pro at position 73, a Cys at position 83 and a Glu at position 98. The signal sequence may be the signal sequence encoded by the aprE gene of B. subtilis.

The Bacillus sp. host cell may have a degU^{Hy}32, oppA, ΔspoIIE3501, ΔaprE, ΔnprE, Δepr, ΔispA, Δbpr, Δvpr, ΔwprA, Δmpr-ybjF, ΔnprB, amyE::xy/RPxylAcomK-ermC genotype and, in certain embodiments, may be a B. subtilis host cell.

The recombinant nucleic acid may be operably linked to a promoter and terminator to form an expression cassette for expression of the SSI protein. The recombinant nucleic acid may be codon optimized for expression of the SSI fusion protein in said Bacillus sp. host cell. The recombinant acid may be present in the genome of the host cell or in a vector that autonomously replicates in the cell.

A culture of cells comprising: a plurality of the above-described Bacillus sp. host cells and culture medium is provided. In particular embodiments, the culture medium may contain the SSI protein.

A method of producing an SSI protein, comprising: culturing the above-described Bacillus cell to provide for secretion of said SSI protein in culture medium is provided. This method may further comprise recovering the SSI protein from the culture medium. The protein may be combined with a laundry detergent to produce a detergent that contains both a subtilisin protease and the SSI protein. The laundry detergent may borax-free laundry detergent.

### BRIEF DESCRIPTION OF THE FIGURES

FIG. 1 shows a map of pKB128-STM-SSI-PG, a vector designed for the expression of SSI as a celA signal sequence-11AG8-SSI fusion protein in Streptomyces cells.

FIG. 2 shows a map of pKB105-SSI-PG, a vector designed for the expression of SSI using the wild type SSI signal sequence of S. albogriseolus, in Streptomyces cells.

FIG. 3 shows a map of pKB105-celA-SSI-PG, a vector designed for the expression of SSI using a celA signal sequence in Streptomyces cells.

FIG. 4 shows an SDS-PAGE gel showing SSI expression in various Streptomyces cells.

FIG. 5 shows a map of pKB65-5′-apraR-3′, a vector designed to inactivate the ssi gene of S. lividans.

FIG. 6 shows a map of p3107-2, a vector designed for the expression of an apreE signal sequence-SSI fusion protein in Bacillus cells.

FIG. 7 shows two SDS-PAGE gels showing SSI expression in various Bacillus cells.

### DETAILED DESCRIPTION

### Definitions

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with general dictionaries of many of the terms used in this invention. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

The term “promoter” is defined herein as a nucleic acid that directs transcription of a downstream polynucleotide in a cell. In certain cases, the polynucleotide may contain a coding sequence and the promoter may direct the transcription of the coding sequence into translatable RNA.

The term “coding sequence” is defined herein as a nucleic acid that, when placed under the control of appropriate control sequences including a promoter, is transcribed into mRNA which can be translated into a polypeptide. A coding sequence may contain a single open reading frame, or several open reading frames separated by introns, for example. A coding sequence may be cDNA, genomic DNA, synthetic DNA or recombinant DNA, for example. A coding sequence generally starts at a start codon (e.g., ATG) and ends at a stop codon (e.g., UAA, UAG and UGA).

The term “recombinant” refers to a polynucleotide or polypeptide that does not naturally occur in a host cell. A recombinant molecule may contain two or more naturally occurring sequences that are linked together in a way that does not occur naturally.

The term “heterologous” refers to elements that are not normally associated with each other. For example, if a host cell produces a heterologous protein, that protein is not normally produced in that host cell. Likewise, a promoter that is operably linked to a heterologous coding sequence is a promoter that is operably linked to a coding sequence that it is not usually operably linked to in a wild-type host cell. The term “homologous” with reference to a polynucleotide or protein, refers to a polynucleotide or protein that occurs naturally in a host cell.

The term “operably linked” refers to an arrangement of elements that allows them to be functionally related. For example, a promoter is operably linked to a coding sequence if it controls the transcription of the sequence, and a signal sequence is operably linked to a protein if the signal sequence directs the protein through the secretion system of a host cell.

The term “nucleic acid” encompasses DNA, RNA, single or doubled stranded and modifications thereof. The terms “nucleic acid” and “polynucleotide” may be used interchangeability herein.

The term “DNA construct” as used herein means a nucleic acid sequence that comprises at least two DNA polynucleotide fragments.

As used herein, the term “reporter” refers to a protein that is easily detected and measured. In certain cases, a reporter may be optically detectable, (e.g., fluorescent, luminescent or colorigenic).

The term “signal sequence” or “signal peptide” refers to a sequence of amino acids at the N-terminal portion of a protein, which facilitates the secretion of the mature form of the protein outside the cell. The mature form of the extracellular protein lacks the signal sequence which is cleaved off during the secretion process.

The term “vector” is defined herein as a polynucleotide designed to carry nucleic acid sequences to be introduced into one or more cell types. Vectors include cloning vectors, expression vectors, shuttle vectors, plasmids, phage or virus particles, DNA constructs, cassettes and the like. Expression vectors may include regulatory sequences such as promoters, signal sequences, a coding sequences and transcription terminators.

An “expression vector” as used herein means a DNA construct comprising a coding is sequence that is operably linked to suitable control sequences capable of effecting expression of a protein in a suitable host. Such control sequences may include a promoter to effect transcription, an optional operator sequence to control transcription, a sequence encoding suitable ribosome binding sites on the mRNA, enhancers and sequences which control termination of transcription and translation.

As used herein, the terms “polypeptide” and “protein” are used interchangeably and include reference to a polymer of any number of amino acid residues. The terms apply to amino acid polymers in which one or more amino acid residue is an artificial chemical analog of a corresponding naturally occurring amino acid, as well as to naturally occurring amino acid polymers. The terms also apply to polymers containing conservative amino acid substitutions such that the polypeptide remains functional. “Peptides” are polypeptides having less than 50 amino acid residues.

A “host cell” is a cell which that contains a recombinant nucleic acid, either in the genome of the host cell or in an extrachromosomal vector that replicates autonomously from the genome of the host cell. A host cell may be any cell type.

“Transformation” means introducing DNA into a cell so that the DNA is maintained in the cell either as an extrachromosomal element or chromosomal integrant.

An “inactivated gene” is a locus of a genome that, prior to its inactivation, was capable of producing a protein, (i.e., capable of being transcribed into an RNA that can be translated to produce a full length, catalytically active, polypeptide). A gene is inactivated when it not transcribed and translated into full length catalytically active protein. A gene may be inactivated by altering a sequence required for its transcription, by altering a sequence required for RNA processing, (e.g., poly-A tail addition), by altering a sequence required for translation, for example. A deleted gene, a gene containing a deleted region, a gene containing a rearranged region, a gene having an inactivating point mutation or frameshift and a gene containing an insertion are types of inactivated genes. A gene may also be inactivated using antisense, RNA interference or any other method that abolishes expression of that gene.

The terms “recovered”, “isolated”, and “separated” as used herein refer to a protein, cell, nucleic acid or amino acid that is removed from at least one component with which it is naturally associated.

As used herein, “culturing” refers to growing a population of microbial cells under suitable conditions in a liquid, solid or semi-solid medium. In one embodiment, culturing refers to fermentative recombinant production of an exogenous protein of interest or other desired end products. Typically, fermentation occurs in a vessel or reactor.

As used herein, the terms “subtilisin” and “subtilisin protein” refer to a serine endopeptidase of the S8 family of peptidases. Subtilisin protein has an activity described as EC 3.4.21.62, according to IUMBM enzyme nomenclature. The activity of exemplary subtilisin proteins is generally described in Philipp et al, (Mol. Cell. Biochem. 1983 51: 5-32).

As used herein, the term “Streptomyces subtilisin inhibitor protein” or “SSI protein”, refers to naturally-occurring SSI protein from Streptomyces albogriseolus (SEQ ID NO:1), and, as will be described in greater detail below, variants of that protein that retain subtilisin protease inhibitory activity. SSI proteins inhibit the protease activity of subtilisin.

Unless otherwise indicated, all amino acid positions in a Streptomyces subtilisin inhibitor protein are relative to SEQ ID NO:1, after alignment of that protein with SEQ ID NO:1 using the BLASTP program (Altschul, Nucl. Acids Res. 1997 25:3389-3402; Schäffer, Bioinformatics 1999 15:1000-1011) under default conditions, as available from the world wide website of the National Center of Biotechnology Information (NCBI).

### SSI Proteins

As noted above, host cells that secrete Streptomyces subtilisin inhibitor (SSI) protein are provided. The subject host cells generally contain a recombinant nucleic acid encoding a fusion protein comprising, in operable linkage, a signal sequence and an SSI protein.

The SSI protein may be a naturally-occurring SSI protein from any species of Streptomyces, (e.g., the SSI protein of Streptomyces albogriseolus (SEQ ID NO:1)) or a variant thereof that retains subtilisin protease inhibitory activity. Examples of variant SSI proteins that retain subtilisin protease inhibitory activity are known and include the approximately 230 SSI proteins listed in Tables 2-10 of WO 00/01826, as well as in Kojima et al., (J. Biochem (1991) 109:377-382), Kojima et al., (Protein Eng. (1990) 3:527-530), Tamura et al., (Biochemistry (1994) 33:14512-14520), Tamura and Sturtevant (J. Mol. Biol. (1995) 249:625-635), Tamura and Sturtevant (J. Mol. Biol. (1995) 249:646-653) Ganz et al., (Protein Eng. Design and Selection (2004) 17:333-339) and WO 98/13387, which references, including any and all SSI protein sequences disclosed therein, are incorporated by reference herein. In certain embodiments, an SSI protein may have an amino acid sequence that is at least 80% identical to SEQ ID NO:1, e.g., at least 85% identical, at least 90% identical, at least 95% identical, at least 97% identical or at least 98% or 99% identical to SEQ ID NO:1. In particular embodiments, the amino acid sequence of an SSI protein may have at least 90% or at least 95% identity to SEQ ID NO:1 and contain the following amino acids substitutions relative to SEQ ID NO:1: A62K, L63I, M73P, D83C and S98E. Accordingly, in particular embodiments, the amino acid sequence of an SSI protein will be at least 90% or at least 95% identical to SEQ ID NO:1 having a Lys at position 62, an Ile at position 63, a Pro at position 73, a Cys at position 83 and a Glu at position 98, where each of these positions in an SSI are defined relative to SEQ ID NO:1 when the SSI protein and SEQ ID NO:1 are aligned using standard sequence alignment methods, e.g., BLASTP (Altschul, Nucl. Acids Res. (1997) 25:3389-3402; Schaffer, Bioinformatics (1999) 15:1000-1011) using default parameters. In a particular embodiment, the SSI protein may have the amino acid sequence of SEQ ID NO:2.

In certain embodiments, the SSI protein produced by the subject host cell may have increased or decreased stability in the presence of a subtilisin protease, as compared to the naturally-occurring SSI protein of Streptomyces albogriseolus (SEQ ID NO:1). In other embodiments, the SSI protein produced by the subject host may have increased or decreased binding affinity for subtilisin protease, as compared to the naturally-occurring SSI protein of Streptomyces albogriseolus (SEQ ID NO:1). Several examples of such SSI proteins are described in the references described in the preceding paragraph.

In one embodiment, the SSI protein may have an amino acid sequence of SEQ ID NO:2 which, as discussed in Ganz, supra, is an SSI protein that is more stable in the presence of subtilisin protease (e.g., BPN′) and has lower affinity to subtilisin protease (e.g., a Kᵢ of 160+/−17 nM) than the naturally-occurring SSI protein of Streptomyces albogriseolus (SEQ ID NO:1).

### Recombinant Nucleic Acids

The recombinant nucleic acid of the subject cells generally contains an expression cassette comprising, in operable linkage: a promoter, a coding sequence encoding a fusion protein comprising a signal sequence and an SSI protein, and a terminator sequence, where the expression cassette is sufficient for the production of the fusion protein in the host cell.

The choice of signal sequence, promoter and terminator largely depend on the host cell used. As noted above, in certain embodiments, a Streptomyces host cell is employed, in which case the signal sequence may be a celA signal sequence. In certain cases, the celA signal sequence may be the signal sequence encoded by the S. lividans cellulase A gene, CelA, as described by Kluepfel et al. (Nature Biotechnol. 1996 14:756-759). In particular embodiments, the amino acid sequence of the celA signal sequence may be that of SEQ ID NO:3. In other embodiments in which a Bacillus host cell is employed, the signal sequence may be any sequence of amino acids that is capable of directing the fusion protein into the secretory pathway of the Bacillus host cell. In certain cases, signal sequences that may be employed include the signal sequences of proteins that are secreted from wild-type Bacillus cells. Such signal sequences include the signal sequences encoded by α-amylase, protease, (e.g., aprE or subtilisin E), or β-lactamase genes. Exemplary signal sequences include, but are not limited to, the signal sequences encoded by an α-amylase gene, an subtilisin gene, an β-lactamase gene, an neutral protease gene (e.g., nprT, nprS, nprM), or a prsA gene from any suitable Bacillus species, including, but not limited to B. stearothermophilus, B. licheniformis, B. lentus, B. subtilis and B. amyloliquefaciens. In one embodiment, the signal sequence is encoded by the aprE gene of B. subtilis (as described in Appl. Microbiol. Biotechnol. 2003 62:369-73). Further signal peptides are described by Simonen and Palva (Microbiological Reviews 1993 57: 109-137), and other references.

Suitable promoters and terminators for use in Bacillus and Streptomyces host cells are known and include: the promoters and terminators of apr (alkaline protease), npr (neutral protease), amy (α-amylase) and β-lactamase genes, as well as the B. subtilis levansucrase gene (sacB), B. licheniformis alpha-amylase gene (amyL), B. stearothermophilus maltogenic amylase gene (amyM), B. amyloliquefaciens alpha-amylase gene (amyQ), B. licheniformis penicillinase gene (penP), B. subtilis xylA and xylB genes, the promoters and terminators described in WO 93/10249, WO 98/07846, and WO 99/43835. Expression cassettes for use in Streptomyces host cells can be constructed using the promoters and terminators described in “Genetic Manipulation of Streptomyces: A Laboratory Manual” (Hopwood et al., Cold Spring Harbor Laboratories, 1985), Hopwood et al., (Hopwood et al., Regulation of Gene Expression in Antibiotic-producing Streptomyces. In Booth, I. and Higgins, C. (Eds) SYMPOSIUM OF THE SOCIETY FOR GENERAL MICROBIOLOGY, REGULATION OF GENE EXPRESSION, Cambridge University Press, 1986 pgs. 251-276), and in Fomwald et al., (Proc. Natl. Acad. Sci. 1987 84: 2130-2134), Pulido et al., (Gene. 1987 56:277-82); Dehottay et al., (Eur. J. Biochem. 1987 166:345-50), Taguchi (Gene. 1989 84:279-86), Schmitt-John et al., (Appl. Microbiol. Biotechnol. 1992 36:493-8), Motamedi (Gene 1995 160:25-31) and Binnie (Protein Expr. Purif. 1997 11:271-8), for example. In one embodiment, the A4 promoter may be employed, which promoter is described in WO 06/054997, which is incorporated by reference herein.

In a particular embodiment, the recombinant nucleic acid may further contain a selectable marker for the selection of cells that contain the recombinant nucleic acid over other cells that do not contain the recombinant nucleic acid. Exemplary selectable markers are described in the references cited in the previous paragraph, and include, but are not limited to, selectable markers that provide resistance to antibiotics, (e.g., resistance to hygromycin, bleomycin, chloroamphenicol, phleomycin, kanamycin, streptomycin, ampicillin, tetracycline, thiostrepton, etc.

In certain embodiments, the coding sequence may be codon optimized for expression of the fusion protein in the host cell used. Since codon usage tables listing the usage of each codon in many cells are known in the art (e.g., Nakamura et al., Nucl. Acids Res. 2000 28: 292) or readily derivable, such nucleic acids can be readily designed giving the amino acid sequence of a protein to be expressed.

A subject recombinant nucleic acid may be present, (e.g., integrated), into a genome (e.g., the nuclear genome) of a host cell, or may be present in a vector, (e.g., a phage, plasmid, viral, or retroviral vector), that autonomously replicates in the host cell. In certain embodiments, the vector may be an expression vector for expressing a protein in a host cell and, as such, may further contain the second expression cassette discussed above.

Vectors systems for expression of recombinant proteins in Streptomyces and Bacillus host cells are well known in the art and need not be discussed in any greater detail than that set forth above.

### Host Cells

A host cell comprising a subject recombinant nucleic acid is also provided. The host cell may be any Bacillus sp. or Streptomyces sp. including, but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis, or S. lividans, S. carbophilus, S. coelocolor, S. rubiginosus, R. albogriseoulus, and S. helvaticus. In some embodiments, the host cell may be a cell of a strain that has a history of use for production of proteins that has GRAS status, (i.e., a Generally Recognized as Safe), by the FDA.

As noted above, in some particularly preferred embodiments, a Bacillus sp. host cell contains nine inactivated protease genes (aprE, nprE, epr, ispA, bpr, vpr, wprA, mpr-ybjF and nprB). In some particular embodiments, the host cell may be a B. subtilis cell having the following genotype: degUHY32, oppA, ΔspoIIE3501, ΔaprE, ΔnprE, Δepr, ΔispA, Δbpr, Δvpr, ΔwprA, Δmpr-ybjF, ΔnprB, amyE::xylRPxylAcomK-ermC. Since the sequence of the entire B. subtilis genome is publicly available and annotated (See e.g., Moszer, FEBS Lett. 1998 430:28-36), the proteases of B. subtilis have been identified and reviewed in detail (See, e.g., He et al., Res. Microbiol. 1991 142:797-803), and gene disruption methods for Bacillus cells are generally well known in the art (See e.g., Lee et al., Applied and Envirommental Microbiology 2000 66: 476-480; Ye et al., Proceedings of the International Symposium on Recent Advances in Bioindustry 1996 pp. 160-169, Seoul, Korea: The Korean Society for Applied Microbiology; Wu et al., J. Bacteriol. 1991 173:4952-4958; and Sloma et al., J. Bacteriol. 1991 173:6889-6895), the construction of such strains is well within the ability of one of skill in the art.

In particular embodiments, a Streptomyces host cell having an inactivated SSI gene may be employed.

In certain embodiments, in addition to containing a recombinant nucleic acid for production of SSI protein, the host cell may further contain a recombinant nucleic acid for expressing a subtilisin protein (i.e., the enzyme inhibited by the SSI protein produced by the cell). As such, in particular embodiments, the cell may contain an expression cassette comprising, in operable linkage: a promoter, a coding sequence encoding a subtilisin protein (which subtilisin protein may be contained in a fusion protein comprising a signal sequence and said subtilisin protein), and a terminator sequence, where the expression cassette is sufficient for the production of the fusion protein in the host cell.

A subtilisin protein may have an amino acid sequence that is found in a wild type genome (i.e., the subtilisin may be a naturally-occurring subtilisin) or may be a variant of a naturally-occurring subtilisin and thus may contain an amino acid sequence that is at least 80%, at least 90%, at least 95% or at least 98% identical to a subtilisin encoded by a wild-type genome. Exemplary subtilisins include: ALCANASE® (Novozymes), FNA™ (Genencor), SAVINASE® (Novozymes), PURFECT™ (Genencor), KAP™ (Kao), EVERLASE™ (Novozymes), PURAFECT OXP™ (Genencor), FN4™ (Genencor), BLAP S™ (Henkel), BLAP X™ (Henkel), ESPERASE® (Novozymes), KANNASE™ (Novozymes) and PROPERASE™ (Genencor). In other embodiments, the subtilisin may be subtilisin 168, subtilisin BPN′, subtilisin Carlsberg, subtilisin DY, subtilisin 147 or subtilisin 309 (See e.g., EP414279B, WO 89/06279 and Stahl et al., J. Bacteriol. 1984 159:811-818). Exemplary subtilisins and other proteases that may be employed herein include those described in WO 99/20770; WO 99/20726; WO 99/20769; WO 89/06279; RE 34,606; U.S. Pat. No. 4,914,031; U.S. Pat. No. 4,980,288; U.S. Pat. No. 5,208,158; U.S. Pat. No. 5,310,675; U.S. Pat. No. 5,336,611; U.S. Pat. No. 5,399,283; U.S. Pat. No. 5,441,882; U.S. Pat. No. 5,482,849; U.S. Pat. No. 5,631,217; U.S. Pat. No. 5,665,587; U.S. Pat. No. 5,700,676; U.S. Pat. No. 5,741,694; U.S. Pat. No. 5,858,757; U.S. Pat. No. 5,880,080; U.S. Pat. No. 6,197,567; and U.S. Pat. No. 6,218,165. Subtilisins in general are reviewed in great detail in Siezen (Protein Sci. 1997 6:501-523), and detergent-additive subtilisins are reviewed in Bryan (Biochim. Biophys. Acta 2000 1543:203-222), Maurer (Current Opinion in Biotechnology 2004 15:330-334) and Gupta (Appl Microbiol Biotechnol. 2002 59:15-32). Certain subtilisins of interest have an activity described as EC 3.4.4.16, according to IUMBM enzyme nomenclature.

In these embodiments, the SSI coding sequence and the subtilisin coding sequence may be parts of different recombinant nucleic acids, or the same recombinant nucleic acid (e.g., on different vectors or on the same vector). In particular embodiments, the SSI protein and the subtilisin may be co-expressed in the cell, such that both proteins are secreted from the host cell into the culture medium.

A culture of cells is provided. In certain embodiments, the culture of cells comprises a plurality of Streptomyces sp. host cells or Bacillus sp. host cells, as described above, and culture medium. The culture medium may comprise SSI protein and, in certain embodiments, the culture medium may comprise both SSI protein and subtilisin protein. In certain embodiments, the SSI protein and the subtilisin protein may be complexed together in the growth medium and, as such, the culture medium may exhibit reduced subtilisin protease activity, as compared to a culture medium in which the same amount of subtilisin protein is present in the absence of SSI protein.

### Protein Production Methods

Methods of using the above-described cells are also provided. In certain embodiments, the subject methods include: culturing a subject cell to produce an SSI protein. In certain embodiments and as discussed above, the protein may be secreted into the culture medium. Particular embodiments of the method include the step of recovering the protein from the culture medium. Depending on whether the host cell contains a recombinant nucleic acid for the expression of a subtilisin protein, the SSI protein may be recovered from the growth medium with or without subtilisin protein. In particular embodiments, the SSI protein and the subtilisin protein may form a complex in the culture medium, and the complex containing the SSI protein and the subtilisin protein may be recovered from the growth medium.

The SSI protein may be recovered from growth media by any convenient method, e.g., by precipitation, centrifugation, affinity, filtration or any other method known in the art. For example, affinity chromatography (Tilbeurgh et al., (1984) FEBS Lett. 16:215); ion-exchange chromatographic methods (Goyal et al., (1991) Biores. Technol. 36:37; Fliess et al., (1983) Eur. J. Appl. Microbiol. Biotechnol. 17:314; Bhikhabhai et al., (1984) J. Appl. Biochem. 6:336; and Ellouz et al., (1987) Chromatography 396:307), including ion-exchange using materials with high resolution power (Medve et al., (1998) J. Chromatography A 808:153; hydrophobic interaction chromatography (Tomaz and Queiroz, (1999) J. Chromatography A 865:123; two-phase partitioning (Brumbauer, et al., (1999) Bioseparation 7:287); ethanol precipitation; reverse phase HPLC; chromatography on silica or on a cation-exchange resin such as DEAE; chromatofocusing; SDS-PAGE; ammonium sulfate precipitation; and gel filtration (e.g., using SEPHADEX G-75), may be employed. In particular embodiments, the detergent-additive protein may be used without purification from the other components the culture medium. In certain embodiments, the components of the culture medium may simply be concentrated, for example, and then used without further purification of the SSI protein (or the SSI protein/subtilisin complex) from the other components of the growth medium.

In some embodiments, a cell cultured may be cultured under batch, fed-batch or continuous fermentation conditions. Classical batch fermentation methods use a closed system, where the culture medium is made prior to the beginning of the fermentation run, the medium is inoculated with the desired organism(s), and fermentation occurs without the subsequent addition of any components to the medium. In certain cases, the pH and oxygen content, but not the carbon source content, of the growth medium may be altered during batch methods. The metabolites and cell biomass of the batch system change constantly up to the time the fermentation is stopped. In a batch system, cells usually progress through a static lag phase to a high growth log phase and finally to a stationary phase where growth rate is diminished or halted. If untreated, cells in the stationary phase eventually die. In general terms, the cells in log phase produce most protein.

A variation on the standard batch system is the “fed-batch fermentation” system. In this system, nutrients (e.g., a carbon source, nitrogen source, sale, O₂, or other nutrient) are only added when their concentration in culture falls below a threshold. Fed-batch systems are useful when catabolite repression is apt to inhibit the metabolism of the cells and where it is desirable to have limited amounts of nutrients in the medium. Measurement of the actual nutrient concentration in fed-batch systems is estimated on the basis of the changes of measurable factors such as pH, dissolved oxygen and the partial pressure of waste gases such as CO₂. Batch and fed-batch fermentations are common and known in the art.

Continuous fermentation is an open system where a defined culture medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth and/or end product concentration. For example, in one embodiment, a limiting nutrient such as the carbon source or nitrogen source is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off may be balanced against the cell growth rate in the fermentation. Methods of modulating nutrients and growth factors for continuous fermentation processes as well as techniques for maximizing the rate of product formation are known.

### Methods of Use

The SSI protein produced using the above described methods may be employed in any product containing a subtilisin protease, including, but not limited to cleaning compositions, (e.g., fabric cleaning compositions (such as laundry detergents), surface cleaning compositions, dish cleaning compositions and automatic dishwasher detergent compositions). Such cleaning compositions are described in great detail in WO0001826, which is incorporated by reference herein. In certain embodiments, the cleaning composition may be a borax-free composition. In particular embodiments, the SSI protein may be complexed with subtilisin protease prior to its addition to the cleaning composition. In other embodiments, the SSI protein may be added to the cleaning composition before, at the same time as, or after addition of the subtilisin protease.

In a particular embodiment, the SSI protein may be employed in an subtilisin-containing laundry detergent comprising from about 1% to 80%, e.g., 5% to 50% (by weight) of surfactant, which may be a non-ionic surfactant, cationic surfactant, an anionic surfactant or a zwitterionic surfactant, or any mixture thereof, e.g., a mixture of anionic and nonionic surfactants. Exemplary surfactants include: alkyl benzene sulfonate (ABS), including linear alkyl benzene sulfonate and linear alkyl sodium sulfonate, alkyl phenoxy polyethoxy ethanol (e.g., nonyl phenoxy ethoxylate or nonyl phenol), diethanolamine, triethanolamine and monoethanolamine. Exemplary surfactants that may be present in laundry detergents are described in U.S. Pat. Nos. 3,664,961, 3,919,678, 4,222,905, and 4,239,659.

The laundry detergent may be in solid, liquid, gel or bar form, and may further contain a buffer such as sodium carbonate, sodium bicarbonate, or detergent builder, bleach, bleach activator, an enzymes, an enzyme stabilizing agent, suds booster, suppresser, anti-tarnish agent, anti-corrosion agent, soil suspending agent, soil release agent, germicide, pH adjusting agent, non-builder alkalinity source, chelating agent, organic or inorganic filler, solvent, hydrotrope, optical brightener, dye or perfumes. The laundry detergent may contain at least a further enzyme, (e.g., another protease, or an amylase, pectate lyase, or lipase), that is protected from subtilisin-induced degradation by the SSI protein.

The subject SSI protein may be employed in any composition useful for cleaning a variety of surfaces in need of peptide stain removal. Such cleaning compositions include detergent compositions for cleaning hard surfaces, unlimited in form (e.g., liquid, gel, bar and granular formation); detergent compositions for cleaning fabrics, unlimited in form (e.g., granular, liquid, gel and bar formulations); dishwashing compositions (unlimited in form); oral cleaning compositions, unlimited in form (e.g., dentifrice, toothpaste, gel and mouthwash formulations); denture cleaning compositions, unlimited in form (e.g., liquid, gel or tablet); and contact lens cleaning compositions, unlimited in form (e.g., liquid, tablet).

The cleaning compositions also comprise, in addition to the proteins described herein, one or more cleaning composition materials compatible with the protease inhibitor. The term “cleaning composition material”, as used herein, means any liquid, solid or gaseous material selected for the particular type of cleaning composition desired and the form of the product (e.g., liquid, granule, bar, spray, stick, paste, gel), which materials are also compatible with the variant used in the composition. The specific selection of cleaning composition materials are readily made by considering the surface material to be cleaned, the desired form of the composition for the cleaning condition during use (e.g., through the wash detergent use). As used herein, “non-fabric cleaning compositions” include hard surface cleaning compositions, dishwashing compositions, oral cleaning compositions, denture cleaning compositions and contact lens cleaning compositions.

The SSI protein can be used with various conventional ingredients to provide fully-formulated hard-surface cleaners, dishwashing compositions, fabric laundering compositions and the like. Such compositions can be in the form of liquids, granules, bars and the like. Such compositions can be formulated as modern “concentrated” detergents which contain as much as 30%-60% by weight of surfactants.

The cleaning compositions herein can optionally, and preferably, contain various anionic, nonionic, zwitterionic, etc., surfactants. Such surfactants are typically present at levels of from about 5% to about 35% of the compositions.

A wide variety of other ingredients useful in detergent cleaning compositions can be included in the compositions herein, including other active ingredients, carriers, hydrotropes, processing aids, dyes or pigments, solvents for liquid formulations, etc. If an additional increment of sudsing is desired, suds boosters such as the C₁₀-C₁₆ alkolamides can be incorporated into the compositions, typically at about 1% to about 10% levels.

A detergent composition can contain water and other solvents as carriers. Low molecular weight primary or secondary alcohols exemplified by methanol, ethanol, propanol, and isopropanol are suitable. Monohydric alcohols are preferred for solubilizing surfactants, but polyols such as those containing from about 2 to about 6 carbon atoms and from about 2 to about 6 hydroxy groups (e.g., 1,3-propanediol, ethylene glycol, glycerine, and 1,2-propanediol) can also be used. The compositions may contain from about 5% to about 90%, typically from about 10% to about 50% of such carriers.

The detergent compositions herein can be formulated such that during use in aqueous cleaning operations, the wash water will have a pH between about 6.8 and about 11.0. Finished products thus are typically formulated at this range. Techniques for controlling pH at recommended usage levels include the use of buffers, alkalis, acids, etc., and are well known to those skilled in the art.

Various bleaching compounds, such as the percarbonates, perborates and the like, can be used in such compositions, typically at levels from about 1% to about 15% by weight. If desired, such compositions can also contain bleach activators such as tetraacetyl ethylenediamine, nonanoyloxybenzene sulfonate, and the like, which are also known in the art. Usage levels typically range from about 1% to about 10% by weight.

Various soil release agents, especially of the anionic oligoester type, various chelating agents, especially the aminophosphonates and ethylenediaminedisuccinates, various clay soil removal agents, especially ethoxylated tetraethylene pentamine, various dispersing agents, especially polyacrylates and polyasparatates, various brighteners, especially anionic brighteners, various suds suppressors, especially silicones and secondary alcohols, various fabric softeners, especially smectite clays, and the like can all be used in such compositions at levels ranging from about 1% to about 35% by weight. Standard formularies and published patents contain multiple, detailed descriptions of such conventional materials.

Enzyme stabilizers may also be used in the cleaning compositions. Such stabilizers include propylene glycol (preferably from about 1% to about 10%), sodium formate (preferably from about 0.1% to about 1%) and calcium formate (preferably from about 0.1% to about 1%).

When formulating the hard surface cleaning compositions and fabric cleaning compositions of the present invention, the formulator may wish to employ various builders at levels from about 5% to about 50% by weight. Typical builders include the 1-10 micron zeolites, polycarboxylates such as citrate and oxydisuccinates, layered silicates, phosphates, and the like. Other conventional builders are listed in standard formularies.

Other optional ingredients include chelating agents, clay soil removal/anti redeposition agents, polymeric dispersing agents, bleaches, brighteners, suds suppressors, solvents and aesthetic agents.

In order to further illustrate the present invention and advantages thereof, the following specific examples are given with the understanding that they are being offered to illustrate the present invention and should not be construed in any way as limiting its scope.

### EXAMPLE 1

### Construction of Strains Expressing Streptomyces Subtilisin Inhibitor as a Cellulase Fusion in Streptomyces lividans

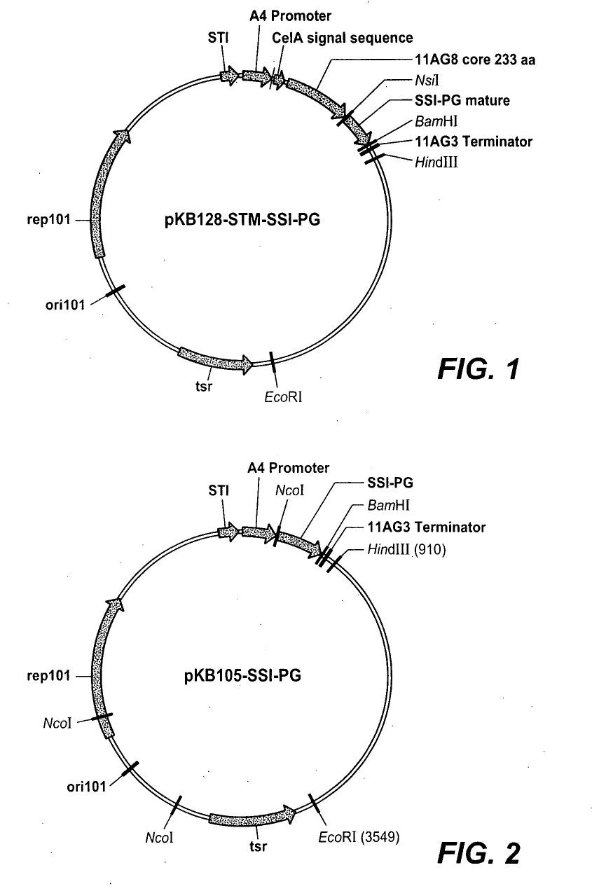

The amino acid sequence of a Streptomyces subtilisin inhibitor (SSI) containing five amino acid substitutions relative to the wild type SSI (A62K L61I M73P D83C S98E; SEQ ID NO:4) was obtained was obtained from Ganz et al (Protein Engineering, Design & Selection 2004 17: 333-339). An DNA encoding this SSI protein (SEQ ID NO:5) was synthesized by DNA2.0 Inc. (Menlo Park, Calif.). Two oligos were designed (SEQ ID NO:6 and SEQ ID NO:7) and used to amplify a DNA fragment encoding the mature SSI-A62K L61I M73P D83C S98E protein (SEQ ID NO:8). A restriction enzyme site NsiI was introduced at the 5′ end of DNA and a restriction enzyme site BamH1 was introduced at the 3′ end of DNA for the cloning purpose. The primers were synthesized by Invitrogen (Carlsbad, Calif.) and they were used in PCR to amplify DNA fragment in a PCR reaction mixture containing DNA, primers, master solution and Q solution (HOT StarTaq, QIAGEN Inc. (Valencia, Calif.)). The PCR reaction was performed in 30 cycles of 98° C. for 30 seconds, 62° C. for 30 seconds and 72° C. for 1 minute and 8 seconds. The final extension at 72° C. was done for 5 minutes and the reaction was chilled to 4° C. The NsiI to BamHI DNA fragment was cloned into Streptomyces expression plasmid pKB128 (SEQ ID NO:9) which was cut with NsiI and BamHI to create expression plasmid pKB128-STM-SSI-PG (FIG. 1). The plasmid pKB128 is identical to plasmid pKB105 except a polylinker sequence containing three restriction enzyme sites: NsiI, MluI and HapI was added before the BamHI site. The expression plasmid (pKB128-STM-SSI-PG) was transformed into Streptomyces lividans strain g3s3 and transformants were selected and grown in TS medium for 2-3 days in the presence of 50 ug/ml thiostrepton at 30° C. Cells were then transferred to medium free of antibiotics, and growth was continued for another three days. An aliquot of the culture was then transferred to a new tube and cells were pelleted by centrifugation. The supernatants were used for enzyme activity assays and protein gel analysis.

One oligo was designed (SEQ ID NO:10) and used to amplify a DNA fragment encoding the mature SSI-A62K L61I M73P D83C S98E protein without first three amino acids (SEQ ID NO:4). A restriction enzyme site NsiI was introduced at the 5′ end of DNA and a restriction enzyme site BamH1 was introduced at the 3′ end of DNA for the cloning purpose. The primers were synthesized by Invitrogen and they were used in PCR to amplify DNA fragment in a PCR reaction mixture containing DNA, primers, master solution and Q solution (HOT StarTaq, Qiagen). The PCR reaction was performed in 30 cycles of 98° C. for 30 seconds, 62° C. for 30 seconds and 72° C. for 1 minute and 8 seconds. The final extension at 72° C. was done for 5 minutes and the reaction was chilled to 4° C. The DNA fragment (NsiI to BamHI fragment, SEQ ID NO:11) was cloned into Streptomyces expression plasmid pKB128 (SEQ ID NO:9) which was cut with NsiI and BamHI to create the expression plasmid (pKB128-STM-SSI-PG short). This plasmid is identical to pKB128-STM-SSI-PG (FIG. 1) except three amino acids at the N-terminal of the mature protein were removed. This plasmid was transformed into Streptomyces lividans strain g3s3 and transformants were selected and grown in TS medium for 2-3 days in the presence of 50 ug/ml thiostrepton at 30° C. Cells were then transferred to medium free of antibiotics, and growth was continued for another three days. An aliquot was transferred to a new tube and the cells were pelleted by centrifugation. The supernatants were used for enzyme activity assay and protein gel analysis.

### EXAMPLE 2

### Construction of Strains Expressing Streptomyces Subtilisin Inhibitor Variant Using Signal Sequence from the SSI Gene

Synthetic DNA sequence (SEQ ID NO:5) encodes the SSI variant of SEQ ID NO:4 operably linked to the SSI signal peptide of S. albogriseolus (SEQ ID NO:12). An NcoI site introduced at the 5′ end of DNA resulted addition of an amino acid glycine residue at position 2. A BamH1 restriction site was also introduced at the 3′ end of DNA for cloning purposes. The synthetic DNA was digested with NcoI and BamHI. The fragment was isolated from the gel and cloned into Streptomyces expression plasmid pKB105 which was also cut with BamHI completely and NcoI partially. The expression plasmid (pKB105-SSI-PG, FIG. 2) was transformed into Streptomyces lividans strain g3s3. The transformants were selected and grown in TS medium for 2-3 days in the presence of 50 ug/ml thiostrepton at 30° C. Cells were then transferred to the production medium free of antibiotics and growth was continued for another three days. An aliquot was transferred to a new tube and the cells were pelleted by centrifugation. The supernatants were used for enzyme activity assays and protein gel analysis.

### EXAMPLE 3

### Construction of Strains Expressing Streptomyces Subtilisin Inhibitor Variant in Streptomyces lividans Using CeIA Signal Sequence

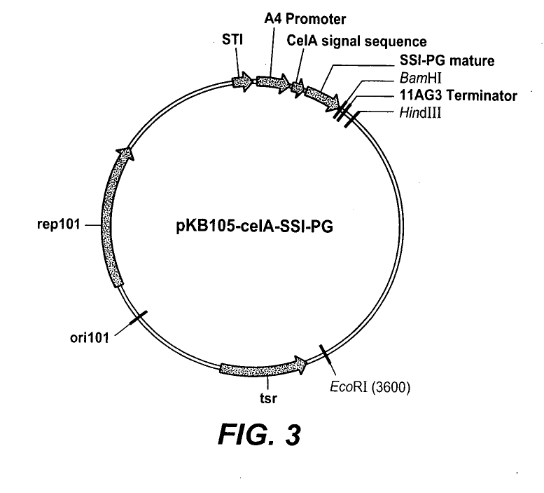

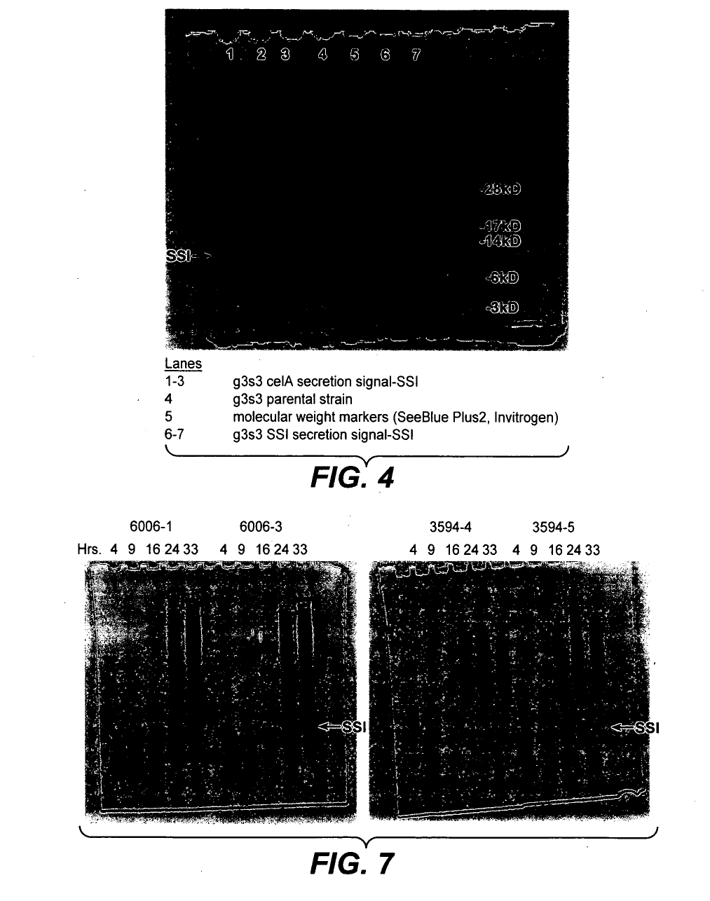

One oligo was designed (SEQ ID NO:13) and used with the oligo (SEQ ID NO:7) to amplify a DNA fragment (SEQ ID NO:14) encoding part of celA signal sequence and the mature SSI-A62K L61I M73P D83C S98E protein. The amino acid sequence of the protein encoded by SEQ ID NO:14 is SEQ ID NO:15. The primers were synthesized by Invitrogen and they were used in PCR to amplify DNA fragment in a PCR reaction mixture containing DNA, primers, master solution and Q solution (HOT StarTaq, Qiagen). The PCR reaction was performed in 30 cycles of 98° C. for 30 seconds, 62° C. for 30 seconds and 72° C. for 1 minute and 8 seconds. The final extension at 72° C. was done for 5 minutes and the reaction was chilled to 4° C. The PCR fragment was digested with restriction enzymes NheI and BamHI. The digested fragment was cloned into Streptomyces expression plasmid pKB105 which was cut with NheI and BamHI to create expression plasmid pKB105-celA-SSI-PG (FIG. 3). The expression plasmid was transformed into Streptomyces lividans strain g3s3 and transformants were selected and grown in TS medium for 2-3 days in the presence of 50 ug/ml thiostrepton at 30° C. Cells were then transferred to the production medium free of antibiotics and growth was continued for another three days. An aliquot was transferred to a new tube and the cells were pelleted by centrifugation. The supernatants were used for enzyme activity assays and protein gel analysis (FIG. 4).

### EXAMPLE 4

### Construction of Strains Containing Deletion of the Streptomyces Subtilisin Inhibitor Gene

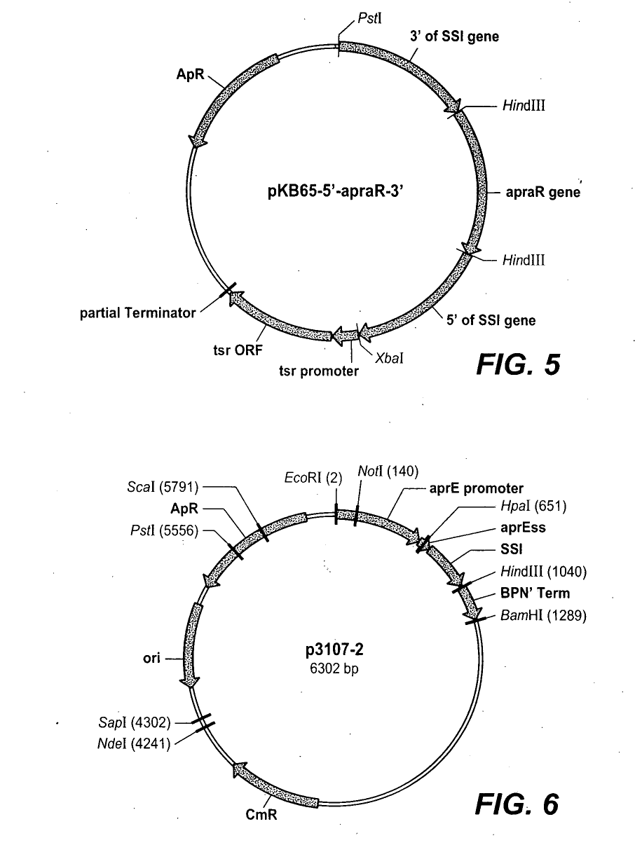

Two oligos were designed (SEQ ID NO:16 and SEQ ID NO:17) and used to amplify the 5′ end of Streptomyces lividans SSI gene. Two other oligos were designed (SEQ ID NO:18 and SEQ ID NO:19) and used to amplify the 3′ end of Streptomyces lividans SSI gene. The primers were synthesized by Invitrogen and they were used in PCR to amplify DNA fragment in a PCR reaction mixture containing DNA, primers, master solution and Q solution (HOT StarTaq, Qiagen). The PCR reaction was performed in 30 cycles of 98° C. for 30 seconds, 62° C. for 30 seconds and 72° C. for 1 minute and 8 seconds. The final extension at 72° C. was done for 5 minutes and the reaction was chilled to 4° C. The 5′ end DNA fragment (SEQ ID NO:20) was cut with XbaI and HindIII and the 3′ end of DNA fragment (SEQ ID NO:21) was cut with HindIII and PstI. They were then cloned into plasmid pKB65 which was cut with XbaI and PstI to create plasmid pKB65-5′/3′ SSI. A HindIII DNA fragment (SEQ ID NO:22) containing apramycin resistant gene was cloned into HindIII site of pKB65-5′/3′ SSI to create an SSI gene deletion plasmid pKB65/SSI5′/apra/3′ (FIG. 5). The plasmid was transformed into Streptomyces lividans strain g3s3.

### EXAMPLE 5

### Construction of Strains Expressing a Streptomyces Subtilisin Inhibitor Variant in Bacillus subtilis

A polynucleotide encoding SSI from Streptomyces albogriseolus having the following amino acid substitutions: A62K, L63I, M73P, D83C, and S98E (Philip J. Ganz, et. al. Protein Eng Des Sel. 2004 17:333-9), codon optimized for expression in Bacillus subtilis was synthesized (SEQ ID NO:23). The 5′ end of the polynucleotide contains a HpaI restriction site (italics) and the first 51 nucleotides of the polynucleotide encodes the C-terminal 17 amino acids of the aprE secretion signal allowing for fusion to the full aprE secretion signal upon ligation. The 3′ end contains the HindIII restriction site immediately following the stop codon of the SSI gene. The SSI protein is encoded by nucleotides 52 to 390 of this sequence. This gene was then synthetically constructed (DNA2.0 Inc., Menlo Park, Calif.).

The amino acid sequence of the protein encoded by this polynucleotide is set forth in SEQ ID NO:24. The protein contains a 17 amino acid aprE secretion signal at the C-terminus.

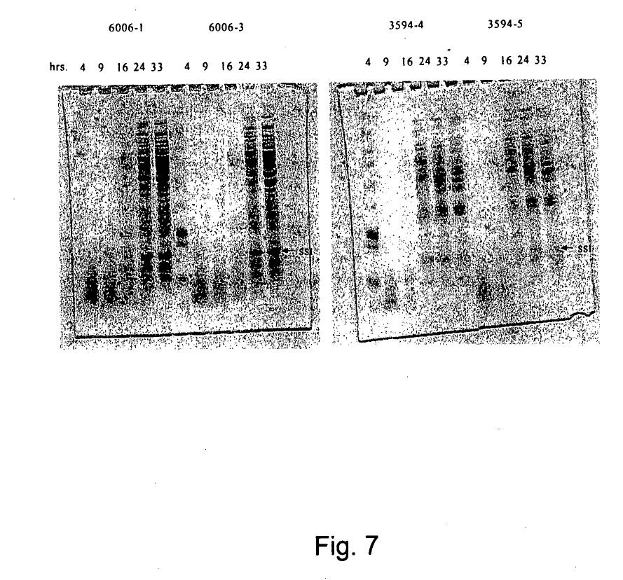

The SSI coding sequence was digested with HpaI and HindIII then ligated to the vector pJHaprEss which was also digested with HpaI and HindIII. The pJHaprEss vector is a pJH101 based vector (Ferrari et al., J. Bacteriol., 152:809-814 [1983]) which allows for replication and selection in E. coli and for selection of integration in Bacillus subtilis. pJHaprEss contains the aprE promoter and secretion signal, and the BPN′ transcriptional terminator. The aprE promoter allows for integration into the Bacillus subtilis genome and drives the expression of the SSI gene. The ligation was transformed into chemically competent E. coli (TOP10, Invitrogen). The resulting plasmid, p3107-2, was isolated from TOP10 E. coli then verified by restriction digest and agarose gel electrophoresis. p3107-2 was then transformed into B. subtilis strains BG3594comK (two protease delete) and BG6006comK (nine protease delete). The B. subtilis strains were made competent by induction of the comK gene under control of a xylose inducible promoter (Hahn et al., Mol. Microbiol., 21:763-775 [1996]). The transformants were selected on Luria Broth agar (LA) plates containing 5 μg/ml chloramphenicol. Several resistant clones were then selected on LA plates containing 25 μg/ml chloramphenicol in order to select for clones that had undergone gene amplification. Two clones of each of the B. subtilis strains BG3594comK(3594-4, 3594-5) and BG6006comK(6006-1, 6006-3) expressing SSI were evaluated in 14L fermentors. Fermentation supernatants were taken at various time points and subjected to SDS PAGE under reducing conditions (FIG. 7).

As can be seen in the gel of FIG. 7, much more SSI is present in the fermentation supernatants of the BG6006comK clones than in the BG3594comK clones. Concentrations of SSI at the 33 hour time points were determined to be:

| Clone # | [SSI] g/L |
|---|---|
| 3594-4 | 1.25 |
| 3594-5 | 1.51 |
| 6006-1 | 4.27 |
| 6006-3 | 3.85 |

The preceding description merely illustrates principles of exemplary embodiments. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A Bacillus sp. host cell comprising:
a recombinant nucleic acid encoding a fusion protein comprising:
a) a signal sequence; and
b) a Streptomyces Subtilisin Inhibitor (SSI) protein;
wherein said host cell secretes said SSI protein from said cell wherein said host cell has inactivated aprE, nprE, epr, ispA, bpr, vpr, wprA, mpr-ybjF and nprB genes.

2. The host cell of claim 1, wherein said SSI protein has increased stability in the presence of subtilisin, as compared to a naturally-occurring SSI protein.

3. The host cell of claim 1, wherein said SSI protein has decreased affinity for subtilisin, as compared to a naturally-occurring SSI protein.

4. The host cell of claim 1, wherein said SSI protein has an amino acid sequence that is at least 95% identical to SEQ ID NO:1, having a Lys at position 62, an Ile at position 63, a Pro at position 73, a Cys at position 83 and a Glu at position 98.

5. The host cell of claim 1, wherein said signal sequence is the signal sequence encoded by the aprE gene of B. subtilis.

6. The host cell of claim 1, wherein said Bacillus sp. host cell has a degU^{Hy} 32, oppA, ΔspoIIE3501, ΔaprE, ΔnprE, Δepr, ΔispA, Δbpr, Δvpr, ΔwprA, Δmpr-ybjF, ΔnprB, amyE::xylRPxylAcomK-ermC genotype.

7. The host cell of claim 1, wherein said Bacillus sp. host cell is a B. subtilis host cell.

8. The host cell of claim 1, wherein said recombinant nucleic acid is operably linked to a promoter and terminator to form an expression cassette.

9. The host cell of claim 1, wherein said recombinant nucleic acid is codon optimized for expression of said SSI fusion protein in said Bacillus sp. host cell.

10. The host cell of claim 1, wherein said recombinant acid is present in the genome of said host cell or in vector that autonomously replicates in said cell.

11. A culture of cells comprising:
a plurality of Bacillus sp. host cells of claim 1; and
culture medium.

12. The culture of cells of claim 1, wherein said culture medium comprises said SSI protein.

13. A method of producing an SSI protein, comprising:
culturing the cell of claim 1 to provide for secretion of said SSI protein in culture medium.

14. The method of claim 13, further comprising recovering said SSI protein from said culture medium.

15. The method of claim 13, further comprising combining said SSI protein with a laundry detergent to produce a detergent that contains that contains both a subtilisin protease and said SSI protein.

16. The method of claim 13, wherein said laundry detergent is borax-free laundry detergent.

17. A Streptomyces sp. host cell comprising:
a recombinant nucleic acid encoding a fusion protein comprising:
a) a celA signal sequence; and
b) a Streptomyces Subtilisin Inhibitor (SSI) protein;
wherein said host cell secretes said SSI protein from said cell.

18. The host cell of claim 17, wherein said SSI protein has increased stability in the presence of subtilisin, as compared to a naturally-occurring SSI protein.

19. The host cell of claim 17, wherein said SSI protein has decreased affinity for subtilisin, as compared to a naturally-occurring SSI protein.

20. The host cell of claim 17, wherein said SSI protein has an amino acid sequence that is at least 95% identical to SEQ ID NO:1, having a Lys at position 62, an Ile at position 63, a Pro at position 73, a Cys at position 83 and a Glu at position 98.

21. A culture of cells comprising:
a plurality of Streptomyces sp. host cells of claim 17; and
culture medium.

22. A method of producing an SSI protein, comprising:
culturing the cell of claim 17 to provide for secretion of said SSI protein in culture medium.

23. The method of claim 22, further comprising combining said SSI protein with a laundry detergent to produce a detergent that contains that contains both a subtilisin protease and said SSI protein.


SEQUENCE LISTING (24 sequences)

SEQ ID NO: 1 - protein, 113 aa, Streptomyces albogriseolus
  wild-type SSI
DAPSALYAPSALVLTVGKGVSATTAAPERAVTLTCAPGPSGTHPAAGSACADLAAVGGDLNALTRGEDVMCPMVYDPVLLTVDGVWQGKRVSYERVFSNECEMNAHGSSVFAF

SEQ ID NO: 2 - protein, 113 aa, Artificial Sequence
  SSI protein containing five amino acid substitutions
DAPSALYAPSALVLTVGKGVSATTAAPERAVTLTCAPGPSGTHPAAGSACADLAAVGGDLNKITRGEDVMCPPVYDPVLLTVCGVWQGKRVSYERVFENECEMNAHGSSVFAF

SEQ ID NO: 3 - protein, 46 aa, Artificial Sequence
  celA signal sequence from S. lividans
MGFGSAPIALCPLRTRRNALKRLLALLATGVSIVGLTALAGPPAQA

SEQ ID NO: 4 - protein, 116 aa, Artificial Sequence
  SSI long form
APGDAPSALYAPSALVLTVGKGVSATTAAPERAVTLTCAPGPSGTHPAAGSACADLAAVGGDLNKITRGEDVMCPPVYDPVLLTVCGVWQGKRVSYERVFENECEMNAHGSSVFAF

SEQ ID NO: 5 - DNA, 448 bp, Artificial Sequence
  SSI coding sequence
ccatgggccggaacaccggcgctgggccatcaccctcggtctcacggccaccgccgtctgcggcccccctctccggggccgcgctcgccgccccgggagatgccccgtccgcgctctacgccccctccgccctggtgctgaccgtcggcaagggcgtcagcgcgacgaccgccgcaccggaacgcgcggtcaccctgacctgtgctccgggcccgtcgggcacccacccggcggccggctcggcctgcgcggacctggccgccgtcggcggcgacctgaacaagatcacgcggggcgaggacgtcatgtgcccgcccgtgtacgacccggtgctgctcaccgtgtgcggcgtctggcagggcaagcgggtctcctacgagcgcgtcttcgagaacgagtgcgagatgaacgcgcacggctcgagcgtcttcgccttctagctggatcc

SEQ ID NO: 6 - DNA, 38 bp, Artificial Sequence
  synthetic oligonucleotide
ggatgcatgccccgggagatgccccgtccgcgctctac

SEQ ID NO: 7 - DNA, 32 bp, Artificial Sequence
  synthetic oligonucleotide
ccggatccagctagaaggcgaagacgctcgag

SEQ ID NO: 8 - DNA, 369 bp, Artificial Sequence
  ssi coding sequence
ggatgcatgccccgggagatgccccgtccgcgctctacgccccctccgccctggtgctgaccgtcggcaagggcgtcagcgcgacgaccgccgcaccggaacgcgcggtcaccctgacctgtgctccgggcccgtcgggcacccacccggcggccggctcggcctgcgcggacctggccgccgtcggcggcgacctgaacaagatcacgcggggcgaggacgtcatgtgcccgcccgtgtacgacccggtgctgctcaccgtgtgcggcgtctggcagggcaagcgggtctcctacgagcgcgtcttcgagaacgagtgcgagatgaacgcgcacggctcgagcgtcttcgccttctagctggatccgg

SEQ ID NO: 9 - DNA, 8948 bp, Artificial Sequence
  vector sequence
ctagagatcgaacttcatgttcgagttcttgttcacgtagaagccggagatgtgagaggtgatctggaactgctcaccctcgttggtggtgacctggaggtaaagcaagtgacccttctggcggaggtggtaaggaacggggttccacggggagagagagatggccttgacggtcttgggaaggggagcttcggcgcgggggaggatggtcttgagagagggggagctagtaatgtcgtacttggacagggagtgctccttctccgacgcatcagccacctcagcggagatggcatcgtgcagagacagacccccggaggtaaccatgggctttgggagcgctcccatcgcgttgtgtccgcttcgcacgaggaggaacgctttgaaacgccttttggccctgctcgcgaccggcgtgtcgatcgtcggcctgactgcgctagccggccccccggcacaggccaaccagcagatctgcgaccgctacggcaccaccacgatccaggaccggtacgtggtgcagaacaaccgctggggcaccagcgccacccagtgcatcaatgtgaccggcaacggtttcgagatcacccaggccgacggttcggtgccgaccaacggcgccccgaagtcctatccctcggtctacgacggctgccactacggcaactgcgcgccccgcacgacgctgcccatgcggatcagctcgatcggcagcgcgcccagcagtgtctcctaccgctacaccggcaacggcgtctacaacgccgcgtacgacatctggctggacccgacaccccgcaccaacggggtgaaccggaccgagatcatgatctggttcaaccgggtcggcccggtccagcccatcggttcgccggtcggcacggcccacgtcggcggccgcagctgggaggtgtggaccggcagcaacggttcgaacgacgtgatctccttcctggcgccctccgcgatcagcagctggagcttcgacgtcaaggacttcgtcgaccaggccgtcagccacggcctggccaccccggactggtacctcaccagcatccaggcgggcttcgagccgtgggagggcggcaccggtctggccgtgaactcgttctcctccgcggtgaacgccgggggcgggaacggcggcactccggggacaccgatgcatacgcgtgttaactgacctggatccgcgagcggatcggctgaccggagcggggaggaggacgggcggccggcggaaaagtccgccggtccgctgaatcgctccccgggcacggacgtggcagtatcagcgccatgtccggcatatcccagccctccgcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcaagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccgaattcggggcatgcctgcaggagtggggaggcacgatggccgctttggtcgacctcaacgagacgatgaagccgtggaacgacaccaccccggcggccctgctggaccacacccggcactacaccttcgacgtctgatcatcactgacgaatcgaggtcgaggaaccgagcgtccgaggaacacaggcgcttatcggttggccgcgagattcctgtcgatcctctcgtgcagcgcgattccgagggaaacggaaacgttgagagactcggtctggctcatcatggggatggaaaccgaggcggaagacgcctcctcgaacaggtcggaaggcccacccttttcgctgccgaacagcaaggccagccgatccggattgtccccgagttccttcacggaaatgtcgccatccgccttgagcgtcatcagctgcataccgctgtcccgaatgaaggcgatggcctcctcgcgaccggagagaacgacgggaagggagaagacgtaacctcggctggccctttggagacgccggtccgcgatgctggtgatgtcactgtcgaccaggatgatccccgacgctccgagcgcgagcgacgtgcgtactatcgcgccgatgttcccgacgatcttcaccccgtcgagaacgacgacgtccccacgccggctcgcgatatcgccgaacctggccgggcgagggacgcgggcgatgccgaatgtcttggccttccgctcccccttgaacaactggttgacgatcgaggagtcgatgaggcggaccggtatgttctgccgcccgcacagatccagcaactcagatggaaaaggactgctgtcgctgccgtagacctcgatgaactccaccccggccgcgatgctgtgcatgaggggctcgacgtcctcgatcaacgttgtctttatgttggatcgcgacggcttggtgacatcgatgatccgctgcaccgcgggatcggacggatttgcgatggtgtccaactcagtcatggtcgtcctaccggctgctgtgttcagtgacgcgattcctggggtgtgacaccctacgcgacgatggcggatggctgccctgaccggcaatcaccaacgcaaggggaagtcgtcgctctctggcaaagctccccgctcttccccgtccgggacccgcgcggtcgatccccgcatatgaagtattcgccttgatcagtcccggtggacgcgccagcggcccgccggagcgacggactccccgacctcgatcgtgtcgccctgagcgtccacgtagacgttgcgtgagagcaggactgggccgccgccgaccgcaccgccctcaccaccgaccgcgaccgcgccatggccgccgccgacggcctggtcgccgccgccgcccgccggttcggcgcctgacccgaccaacccccgcggggcgccggcacttcgtgctggcgccccgcccccacccaccaggagaccgaccatgaccgacttcgacggacgcctgaccgaggggaccgtgaacctggtccaggaccccaacggcggtggctggtccgcccactgcgctgagcccggttgcgactgggccgacttcgccggaccgctcggcttccagggcctcgtggccatcgctcgccgacacacgcactgaccgcacgtcaaagccccgccggatacccggcggggctctcttcggccctccaagtcacaccagccccaaggggcgtcgggagtggcggagggaacctctggcccgattggtgccaggattcccaccagaccaaagagcaacgggccggacttcgcacctccgacccgtccgctcccagactcgcgccccttagccgggcgagacaggaacgttgctcgtgcccagagtacggagcgatgccgaggcattgccagatcggcccgccgggccccgctgccactgcgggaccgcaattgcccacacaccgggcaaacggccgcgtatctactgctcagaccgctgccggatggcagcgaagcgggcgatcgcgcgtgtgacgcgagatgccgcccgaggcaaaagcgaacaccttgggaaagaaacaacagagtttcccgcacccctccgacctgcggtttctccggacggggtggatggggagagcccgagaggcgacagcctctgggaagtaggaagcacgtcgcggaccgaggctgcccgactgcggaaagccgcccggtacagccgccgccggacgctgtggcggatcagcggggacgccgcgtgcaagggctgcggccgcgccctgatggaccctgcctccggcgtgatcgtcgcccagacggcggccggaacgtccgtggtcctgggcctgatgcggtgcgggcggatctggctctgcccggtctgcgccgccacgatccggcacaagcgggccgaggagatcaccgccgccgtggtcgagtggatcaagcgcggggggaccgcctacctggtcaccttcacggcccgccatgggcacacggaccggctcgcggacctcatggacgccctccagggcacccggaagacgccggacagcccccggcggccgggcgcctaccagcgactgatcacgggcggcacgtgggccggacgccgggccaaggacgggcaccgggccgccgaccgcgagggcatccgagaccggatcgggtacgtcggcatgatccgcgcgaccgaagtcaccgtggggcagatcaacggctggcacccgcacatccacgcgatcgtcctggtcggcggccggaccgagggggagcggtccgcgaagcagatcgtcgccaccttcgagccgaccggcgccgcgctcgacgagtggcaggggcactggcggtccgtgtggaccgccgccctgcgcaaggtcaaccccgccttcacgcccgacgaccggcacggcgtcgacttcaagcggctggagaccgagcgcgacgccaacgacctcgccgagtacatcgccaagacccaggacgggaaggcgcccgccctcgaactcgcccgcgccgacctcaagacggcgaccggcgggaacgtcgccccgttcgaactcctcggacggatcggggacctgaccggcggcatgaccgaggacgacgccgccggggtcggctcgctggagtggaacctctcgcgctggcacgagtacgagcgggcaacccggggacgccgggccatcgaatggacccgctacctgcggcagatgctcgggctcgacggcggcgacaccgaggccgacgacctcgatctgctcctggcggccgacgccgacggcggggagctgcgggccggggtcgccgtgaccgaggacggatggcacgcggtcacccgccgcgccctcgacctcgaggcgacccgggccgccgaaggcaaggacggcaacgaggattcggcggccgtgggcgaacgggtgcgggaggtcctggcgctggccgacgcggccgacacagtggtggtgctcacggcgggggaggtggccgaggcgtacgccgacatgctcgccgccctcgcccagcgccgcgaggaagcaactgcacgccgacggcgagagcaggacgacgaccaggacgacgacgccgacgaccgccaggagcgggccgcccggcacatcgcccggctcgcaagtgggcccacttcgcactaactcgctcccccccgccgtacgtcatcccggtgacgtacggcgggggtcggtgacgtacgcggcgacggcggccggggtcgaagccgcgggagtaatcctgggattactcgcccggggtcggccccgccggcacttcgtgcaggcggtacctcgcgcccgactcgcctcgctacgagacgtgccgcgtacggtcgtcggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggctgcagctcgcccggccgccggtccccctgccgtccggcccgtcccagaggcagcgtcggcggctcctgcctcccccgcccggcgccgaccgggacccgcaaaccccttgatccgctgtcgggggtgatcactaggtgggtgccgaagtgatcacggggaggactgatgcaccaccaggaccgggaccaggaccaggcgttagcggcagtgctggccgcactgctcctggtcggcgggacgctgatcgtgcgggagctcctgggcctgtggcccgccgtggcggtcggcatggcgcccgccctcgccctctacggaggcccgcccgcggcccgccggatagccgtcgcggtcgaggtccgccggttccgccggcatcttgcccaccacgatcgggcagccggatgaccggccacgacggagccgcacggctgaccagctcgacggccgccacctcatcgcggcagcaggtgctccccagcaacccacgacggggctcagggtcgcctcacgcggctcagcaccgcgacggcgggggtacggcgctccgggaggctgacaggcgctcagacggccgcgtagggccgcgagtcccccacccctccccgctgccctgtcggcgagcacaacggcgatgcccgcagtcggcggagcaggcgccacgtaaaccgcccaccgatgccgcccccgtcgtgtgcgcgggccggtcggcggccgggccggagcggggcgaagacaggagcgtcggccgggccgtgggccgggccgcgcggcccgctcgcgggccgccttgatgacgtagggaaagttgtaccgcaaaaaacgcagcctgaactagttgcgatcct

SEQ ID NO: 10 - DNA, 38 bp, Artificial Sequence
  synthetic oligonucleotide
ggatgcatgatgccccgtccgcgctctacgccccctcc

SEQ ID NO: 11 - DNA, 360 bp, Artificial Sequence
  polynucleotide encoding SEQ ID NO1
ggatgcatgatgccccgtccgcgctctacgccccctccgccctggtgctgaccgtcggcaagggcgtcagcgcgacgaccgccgcaccggaacgcgcggtcaccctgacctgtgctccgggcccgtcgggcacccacccggcggccggctcggcctgcgcggacctggccgccgtcggcggcgacctgaacaagatcacgcggggcgaggacgtcatgtgcccgcccgtgtacgacccggtgctgctcaccgtgtgcggcgtctggcagggcaagcgggtctcctacgagcgcgtcttcgagaacgagtgcgagatgaacgcgcacggctcgagcgtcttcgccttctagctggatccgg

SEQ ID NO: 12 - protein, 145 aa, Artificial Sequence
  substituted SSI and native SSI signal sequence
MGRNTGAGPSPSVSRPPPSAAPLSGAALAAPGDAPSALYAPSALVLTVGKGVSATTAAPERAVTLTCAPGPSGTHPAAGSACADLAAVGGDLNKITRGEDVMCPPVYDPVLLTVCGVWQGKRVSYERVFENECEMNAHGSSVFAF

SEQ ID NO: 13 - DNA, 48 bp, Artificial Sequence
  synthetic oligonucleotide
aagctagccggccccccggcacaggccgccccgggagatgccccgtcc

SEQ ID NO: 14 - DNA, 388 bp, Artificial Sequence
  polynucleotide encoding celA-SSI fusion
aagctagccggccccccggcacaggccgccccgggagatgccccgtccgcgctctacgccccctccgccctggtgctgaccgtcggcaagggcgtcagcgcgacgaccgccgcaccggaacgcgcggtcaccctgacctgtgctccgggcccgtcgggcacccacccggcggccggctcggcctgcgcggacctggccgccgtcggcggcgacctgaacaagatcacgcggggcgaggacgtcatgtgcccgcccgtgtacgacccggtgctgctcaccgtgtgcggcgtctggcagggcaagcgggtctcctacgagcgcgtcttcgagaacgagtgcgagatgaacgcgcacggctcgagcgtcttcgccttctagctggatccgg

SEQ ID NO: 15 - protein, 162 aa, Artificial Sequence
  celA-SSI fusion
MGFGSAPIALCPLRTRRNALKRLLALLATGVSIVGLTALAGPPAQAAPGDAPSALYAPSALVLTVGKGVSATTAAPERAVTLTCAPGPSGTHPAAGSACADLAAVGGDLNKITRGEDVMCPPVYDPVLLTVCGVWQGKRVSYERVFENECEMNAHGSSVFAF

SEQ ID NO: 16 - DNA, 27 bp, Artificial Sequence
  synthetic oligonucleotide
ggtctagaggccgggtccgagaagtag

SEQ ID NO: 17 - DNA, 31 bp, Artificial Sequence
  synthetic oligonucleotide
ggaagcttgtgtgcatccttccgctcgtttc

SEQ ID NO: 18 - DNA, 27 bp, Artificial Sequence
  synthetic oligonucleotide
ggaagcttctgagggaccgggaccgcc

SEQ ID NO: 19 - DNA, 26 bp, Artificial Sequence
  synthetic oligonucleotide
aactgcaggaggacaccgccctgctg

SEQ ID NO: 20 - DNA, 1009 bp, Streptomyces lividans
ggtctagaggccgggtccgagaagtagttggcggtgccgacgccgtgcaggaacggcaggtacgcctggtcgaggtcgcgcgggcgccaggacagcagcggcgtgtccagcgtgacgaacagcgccgtgtacccggccgcccgggcccggtccaggaagctgcgcgccacctcgcggtccttcggccagtacagctggaaccagcgctcggcgtcgcccatcgcctcggcgacctcctccatgggcgtgctggaggcggaggagaggatgtacggcacaccctgggccgcggcggcccgggcggccgcgggctccgcctccgggtgcatgatcgacagcacaccgaccggcgcgagcgccagcggggcgggcagggaacggcccagcacctcgacacgcaggtcgcgttcgtgcacgtcccgcagcatgcgcggaacgatccggcgcctggccagcgccgcccggttggctcgggcggtgctgccgtcgcccgcgctgcccgcgacgtatccgaaggggccgggcccgaggcgccgctcggtcagctcctcgagccgggtcaggtcggtgggcagccgcgggaccgcgcccgtcatgccgttcagatagatctcgtactggaaatccgcccagtgcttcggcatccccggtccgcctctcgtcgtcgagctcgcgctgcccggcaccatacccgcgagtatgccgggggtggcggtcggggccgacggacctgttccgggggtgtttcaggcctattggcgaaggtgcggatcacgccattcggccagttctgttctcaataatcgatcaagaaacgcccagttccggacttggaacgttctaattctgtgacttcacgccactgattcaatacgcaaggttaccgaaacccgtggggtcgagatgagtttcggtgcgggactcggcagactcgcgctcgccgctccggcaccgacgggtcggaccggcaccaccctcgaaacgagcggaaggatgcacacaagcttcc

SEQ ID NO: 21 - DNA, 1009 bp, Streptomyces lividans
ggaagcttctgagggaccgggaccgccgggaccgtgcggcgtgatcggctgctcgctactggggagtgcgagcgccgccgtacgggtcccgcgggcccgcaccggggacggcggacggagtggggccgtccgccgttctcccggttcaggggcacggtcggccgttcgcggccgggccgtccgcggccctggcggaccgggcacggtggctggtgtcgcaccacggatagcgccgactgcgccggcacgtgcacagcgccacccggaagcggtcggaggacaccgtcgtcccgtcctccagctccacctccaccgggccttccaccagcagcggcccccggcgctggaccttgatccggcgccgggcgtcggacgggcgcgcgggctcagacggggagttcggcacggacgaccaccagctcttcctcgtagtccgccagggacagcagcccgcgctgccgcagccagcgctcccggccgcgcaccacgggaccgaacgcgatccgccgccgccgggtcaccgccgccttcagccccgcctcgcgcagcagcgcctcggtcctcgcggggtcactgagcgccgactgcacgagcagcagcaccccgccggggcgcagcagccggggtacttccaggcagatccggtccaggaccatgcgaccgtcgtggcccgcgtcccaggcccgcgcggcaccacgcggccggcggccggtggcgggtgccggcacatacggcggattggccagcaccaggtcgaacgactcgccgcgcacgggtgtgaagaggttgccgtgccggacgcggatccgcagcccggcccgggccgcgttgagccgcgcggcgcacaccgcgcgccacgacacgtccaccgcggtcacccggccgccccgcccggccgcagccagggcgagggcgcccgagccggttcccacgtcgagcacggcggcgcccggcgggagcgactcgtcggacagcgccccggccagcagggcggtgtcctcctgcagtt

SEQ ID NO: 22 - DNA, 1085 bp, Streptomyces lividans
aagcttgaagaaagacaatccccgatccgctccacgtgttgccccagcaatcagcgcgaccttgcccctccaacgtcatctcgttctccgctcatgagctcagccaatcgactggcgagcggcatcgcattcttcgcatcccgcctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagaagctt

SEQ ID NO: 23 - DNA, 399 bp, Artificial Sequence
  SSI polynucleotide codon optimized for Bacillus
gcgttaacgttaatctttacgatggcgttcagcaacatgagcgcgcaggctgatgctcctagtgccttgtacgcaccgtctgccttggtcttaactgtcggcaagggcgtatcagctaccacagctgcgccggaacgtgcggtcaccctgacatgtgctcctggaccgtccggcactcatcctgctgctggtagcgcgtgtgctgaccttgcggccgttggcggggacttaaacaaaatcacgagaggagaagatgtcatgtgcccgccggtgtacgatcctgtgcttttgacggtttgcggtgtttggcaaggcaaacgggtttcttatgagcgggtgttcgaaaacgagtgtgaaatgaatgctcatggttcttctgttttcgccttttgaaagctt

SEQ ID NO: 24 - protein, 130 aa, Artificial Sequence
  aprE-SSI fusion
ALTLIFTMAFSNMSAQADAPSALYAPSALVLTVGKGVSATTAAPERAVTLTCAPGPSGTHPAAGSACADLAAVGGDLNKITRGEDVMCPPVYDPVLLTVCGVWQGKRVSYERVFENECEMNAHGSSVFAF